(19) United States
(12) Patent Application Publication
(10) Pub. No.: US 2025/0329071 A1
(43) Pub. Date: Oct. 23, 2025

(54) SYSTEMS AND METHODS FOR FOUR-DIMENSIONAL FLOW MRI DATASETS

(71) Applicant: Purdue Research Foundation, West Lafayette, IN (US)

(72) Inventors: Pavlos P. Vlachos, West Lafayette, IN (US); Jiacheng Zhang, Brookfield, WI (US)

(73) Assignee: Purdue Research Foundation, West Lafayette, IN (US)

(21) Appl. No.: 18/870,924

(22) PCT Filed: Apr. 3, 2023
(86) PCT No.: PCT/US2023/017269

§ 371 Date: Dec. 2, 2024

Related U.S. Application Data
(60) Provisional application No. 63/348,723, filed on Jun. 3, 2022.

(51) Int. Cl. G06T 11/00; G06T 7/20; G16H 50/70

(52) U.S. Cl. CPC G06T 11/008; G06T 7/20; G16H 50/70; G06T 2207/10076; G06T 2207/10088; G06T 2207/30104; G06T 2210/41

(57) ABSTRACT

A method of processing data by an imaging system is described. The imaging system generates a velocity data set and magnitude data set representative of a fluid. The method includes receiving velocity data set from the imaging system, calculating a phase variation data set from a wrapped phase field data set associated with the velocity data set, calculating a phase difference uncertainty data set from the magnitude data set, using the phase variation-data set and the phase difference uncertainty data set, performing a computational reconstruction of the phase field, data set to generate an unwrapped phase data set, converting the unwrapped phase to a first velocity field data set; and outputting a resultant velocity field set based upon the first velocity field data set.

### CROSS-REFERENCE TO RELATED APPLICATIONS

This application is related to and claims the priority benefit of U.S. Provisional Application No. 63/348,723, entitled “Systems and Methods for Processing Four-Dimensional Flow MRI Datasets,” filed Jun. 3, 2022, the contents of which are hereby incorporated by reference in their entirety into the present disclosure.

### GOVERNMENT SUPPORT CLAUSE

This invention was made with government support under EB025766, HL115267, and NS106696 awarded by the National Institutes of Health. The government has certain rights in the invention.

### TECHNICAL FIELD

The present application relates to medical imaging, and specifically to medical imaging methods to measure blood flow and evaluate hemodynamic quantities.

### BACKGROUND

This section introduces aspects that may help facilitate a better understanding of the disclosure. Accordingly, these statements are to be read in this light and are not to be understood as admissions about what is or is not prior art.

Magnetic Resonance Imaging (MRI) is most commonly employed in medical imaging, although can be used in other fields. MRI machines include a main magnet which is typically an annular array of coils having a central or longitudinal bore. The main magnet is capable of producing a strong stable magnetic field (e.g., 0.5 Tesla to 3.0 Tesla). The bore is sized to receive at least a portion of an object to be imaged, for instance a human body. When used in medical imaging applications, the MRI machine may include a patient table which allows a prone patient to be easily slid or rolled into and out of the bore.

MRI machines also include gradient magnets. The gradient magnets produce a variable magnetic field that is relatively smaller than that produced by the main magnet (e.g., 180 Gauss to 270 Gauss), allowing selected portions of an object (e.g., patient) to be imaged. MRI machines also include radio frequency (RF) coils which are operated to apply radiofrequency energy to selected portions of the object (e.g., patient) to be imaged. Different RF coils may be used for imaging different structures (e.g., anatomic structures). For example, one set of RF coils may be appropriate for imaging a neck of a patient, while another set of RF coils may be appropriate for imaging a chest or heart of the patient. MRI machines commonly include additional magnets, for example resistive magnets and/or permanent magnets.

The MRI machine typically includes, or is communicatively coupled to, a computer system used to control the magnets and/or coils and/or to perform image processing to produce images of the portions of the object being imaged. Conventionally, MRI machines produce magnitude data sets which represent physical structures, for instance anatomical structures. The data sets are often conform to the Digital Imaging and Communications in Medicine (DICOM) standard. DICOM files typically include pixel data and metadata in a prescribed format.

Four-dimensional flow MRI (collectively referred to herein as “4D Flow MRI”) is an advanced MRI technique which allows for in vivo acquisition of time-resolved three-dimensional (3D) blood flow, thus enabling quantitative analysis of volumetric, time varying hemodynamic quantities such as flow rates, wall shear stress (WSS), pressure, etc. 4D flow MRI has demonstrated great potential to improve the diagnostics of cardiovascular and cerebrovascular diseases.

### SUMMARY

Several systems, methods, and algorithms have been proposed for the pre- and post-processing analysis of 4D flow MRI data; however, they are either untested or unreliable. Accordingly, improvements for 4D Flow MRI systems are needed. The present disclosure includes aspects which can overcome the limitations of existing 4D Flow MRI systems. Generally, the present disclosure introduces and evaluates a robust and reliable phase unwrapping method for 4D flow MRI.

As described in various embodiments herein, methods of processing data generated by an imaging system can include various steps. The imaging system an be operable to generate a velocity data set and a magnitude data set representative of a fluid flow. Thus, steps can include one or more of receiving the velocity data set from the imaging system, calculating a phase variation data set from a wrapped phase field data set associated with the velocity data set, and calculating a phase difference uncertainty data set from the magnitude data set. Next, using the phase variation data set and the phase difference uncertainty data set, steps can include performing a computational reconstruction of the phase field data set to generate an unwrapped phase data set, converting the unwrapped phase to a first velocity field data set, and outputting a resultant velocity field set based upon the first velocity field data set.

In some embodiments, the method can further include outputting the resultant velocity field set includes transmitting the resultant velocity field set to a graphical display. In some embodiments, performing the computational reconstruction of the phase field data set to generate the unwrapped phase data set can include performing a weighted least squares operation to generate the unwrapped phase data set. In other embodiments, the phase variation data set can include a spatial phase variation component and a temporal phase variation component, wherein the spatial phase variation component can be representative of the difference between two or more neighboring voxels and the temporal phase variation component is representative of the difference between two or more consecutive cardiac frames.

In some embodiments, converting the unwrapped phase to the first velocity field data set can include multiplying the unwrapped phase by (venc/π). In other embodiments, calculating the phase variation data set from the wrapped phase field value can include applying the formula [symbol] = [symbol] (Δψ), wherein [symbol] is the phase variation data set, wherein Δψ is a spatial variation data set or a temporal variation data set of the wrapped phase field data set. In still other embodiments, calculating the phase difference uncertainty data set from the magnitude data set can include incorporating with the magnitude data set a divergence-free constraint of incompressible flow.

In some embodiments, outputting the resultant velocity field set based upon the first velocity field data set can include calculating a second phase variation data set from a second wrapped phase field data set associated with the first velocity field data set and calculating a second phase difference uncertainty data set from a second magnitude data set associated with the first velocity field data set. Next, using the second phase variation data set and the second phase difference uncertainty data set, the steps can include performing a second computational reconstruction of the second phase field data set to generate a second unwrapped phase data set, converting the second unwrapped phase to a second velocity field data set, and outputting the resultant velocity field set based upon the second velocity field data set.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein does not necessarily address every object described herein, and each embodiment does not necessarily include each feature described. Other forms, embodiments, objects, advantages, benefits, features, and aspects of the present disclosure will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1A depicts a flowchart representation of one exemplary method of phase unwrapping using constrained weighted least-squares (CWLS);

FIG. 1B depicts a schematic diagram showing one exemplary sequence of temporal phase unwrapping from the time point with lowest average velocity (at t₀) to the time point with highest average velocity (at t_{N}) along the forward and backward directions in a cyclic manner, with the waveform demonstrating the flow rate in one cardiac cycle;

FIG. 2A depicts an experimental output diagram for one experimental setup, showing a u velocity field at peak diastole on the center x-z plane;

FIG. 2B depicts an experimental output diagram for the experimental setup, showing a u-phase at peak diastole with VR=0.2 and SNR_{I}=10;

FIG. 2C depicts an experimental output diagram for the experimental setup, showing the magnitude intensity fields at peak diastole with VR=0.2 and SNR_{I}=10;

FIG. 2D depicts an experimental output diagram for the experimental setup, showing the resulting u velocity fields on the center x-z plane at peak diastole unwrapped with CWLS;

FIG. 2E depicts an experimental output diagram for the experimental setup, showing the resulting u velocity fields on the center x-z plane at peak diastole unwrapped with 4D Lap;

FIG. 3A depicts a data table for the experimental setup, showing the unwrapping success rates (SRs) of CWLS and 4D Lap methods of the synthetic phase datasets of LV flow;

FIG. 3B depicts a data table for the experimental setup, showing the VELs (by %) for the resulting velocity fields by CWLS and 4D Lap methods;

FIG. 4A depicts graphical representations for the experimental setup, showing SRs and Vₑᵣᵣₒᵣₛ (by %) of the four different unwrapping methods with or without the uncertainty-based weighting and the divergence-free regularization for the synthetic cases with VRs from 0.2 to 1.0 and an SNR of 5;

FIG. 4B depicts graphical representations for the experimental setup, showing SRs and Vₑᵣᵣₒᵣₛ (by %) of the four different unwrapping methods with or without the uncertainty-based weighting and the divergence-free regularization for the synthetic cases with VRs from 0.2 to 1.0 and an SNR of 10;

FIG. 4C depicts graphical representations for the experimental setup, showing SRs and Vₑᵣᵣₒᵣₛ (by %) of the four different unwrapping methods with or without the uncertainty-based weighting and the divergence-free regularization for the synthetic cases with VRs from 0.2 to 1.0 and an SNR of 20;

FIG. 4D depicts graphical representations for the experimental setup, showing SRs and Vₑᵣᵣₒᵣₛ (by %) of the four different unwrapping methods with or without the uncertainty-based weighting and the divergence-free regularization for the synthetic cases with VRs from 0.2 to 1.0 and an SNR of 50;

FIG. 5A depicts an experimental output diagram for the experimental setup, showing the intensity magnitude fields on a plane along the centerline of the pipe;

FIG. 5B depicts an experimental output diagram for the experimental setup, showing the w velocity phase fields from acquisitions with vencs of 4 cm/s, 8 cm/s, and 16 cm/s, respectively, the fields being shown on a plane along the centerline of the pipe;

FIG. 6A depicts a graphical representation for the experimental setup, showing boxplots of the statistical distributions of SRs from 22 artificially wrapped datasets for each VR, the centerline of each box indicating the median, the edges indicating the 25th and 75th percentiles;

FIG. 6B depicts an experimental output diagram for the experimental setup, showing the u phase fields on the center x-y plane at peak systole of an artificially wrapped BAV dataset with VR=0.3;

FIG. 6C depicts an experimental output diagram for the experimental setup, showing the u phase fields on the center x-y plane at peak systole with real-aliasing for one BAV dataset;

FIG. 6D depicts an experimental output diagram for the experimental setup, showing one TAV-AA dataset where the patient additionally had a repaired coarctation causing a high-speed jet in the proximal descending aorta; and

FIG. 7 depicts a block diagram of one exemplary system configured for phase unwrapping of imaging data using flow-physics constrained computations.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown, or the precise experimental arrangements used to arrive at the various graphical results shown in the drawings.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Overview

4D flow MRI is based on the phase contrast (PC) technique which is a type of MRI technique used to visualize and measure the motion of fluids, such as blood flow, along all dimensions within the body. This technique uses the phase shift of the MR signal caused by the motion of the fluid relative to the surrounding tissue to create an image. A pair of gradient pulses are applied to the magnetic field during the imaging sequence and these pulses cause a phase shift in the MR signal that is proportional to the velocity of the fluid. By adjusting the timing and strength of the gradient pulses, it is possible to separate the signal from moving fluids from that of stationary tissue. The resulting phase contrast images show the velocity of the fluid as a bright or dark signal, superimposed on an anatomical image of the surrounding tissue. Accordingly, the technique can be used to measure the velocity of blood flow in arteries and veins, as well as in other types of fluids within the body.

For the PC technique, a predefined velocity encoding sensitivity parameter (venc) determines the maximum and minimum velocity that can be recorded in the phase data as π and −π, respectively. Therefore, the velocity field can be obtained by multiplying the phase with venc/π. Whenever a velocity component is greater than venc or lower than −venc, the acquired phase is wrapped and leads to velocity aliasing (i.e., the velocity of the fluid exceeds the maximum measurable velocity of the imaging sequence). To avoid aliasing, the venc is suggested to be set approximately 10% higher than the maximum expected velocity. However, high venc leads to high noise level since the velocity-to-noise ratio (VNR) is inversely proportional to venc.

One strategy to capture the wide dynamic range associated with physiologic blood flow while maintaining the low noise level associated with low venc data is to perform acquisitions with a set of two or more vencs. The acquired high-venc data can then be employed for unwrapping the low-venc data. However, despite the efforts to accelerate the multi-venc acquisition, the total scan time is still unavoidably longer than a single scan, which is a limitation of the approach. Another strategy is algorithmically unwrapping the wrapped phase data. Several methods and algorithms have been proposed for 4D flow MRI. However, these methods and algorithms are either untested or unreliable for low-venc acquisitions with large-aliased areas or repeatedly wrapped regions. Phase noise also dramatically affects the performances of the unwrapping algorithms.

The present disclosure introduces and evaluates a robust and reliable phase unwrapping method for 4D flow MRI. The proposed method, flow-physics constrained weighted least-squares (CWLS), incorporates the divergence-free constraint of incompressible flow with the estimated phase variations to formulate an optimization problem. The unwrapped phase may be obtained using CWLS with weights generated based on the phase variation uncertainty. CWLS also utilizes the temporal phase information to enhance the robustness by unwrapping from timepoints least likely to be wrapped towards those likely to be wrapped. As described below, the CWLS method was tested and selected results are provided using synthetic phase data of left ventricular (LV) flow and in vitro Poiseuille flow measured using 4D flow MRI. The method is then applied to in vivo aortic 4D flow MRI data from 30 subjects. Additionally, the performance of the proposed method was compared to the state-of-the-art 4D single-step Laplacian algorithm (hereinafter “4D Lap”). While a weighted least-squares computational method is described herein for performing computational reconstruction of phase field data to generate unwrapped phase data, it should be understood that various other computational reconstruction methods may be used such as by using a regression model or an artificial-intelligence (AI) based model.

### II. Theory

Phase wrapping in 4D flow MRI can be presented as:

$$ψ = ( ϕ ) = ϕ + 2 n π with n = - round ( \frac{ϕ}{2 π} ) ∈ Z ,$$

(hereinafter “Equation 1”) where ψ is the wrapped phase, ϕ is the unwrapped phase, [symbol] represents the wrapping operation which adds a multiple of 2π to ϕ such that ψ is within the range (−π, π), round [symbol] means rounding to the nearest integer, and Z is the set of integers. ϕ is related with the underlying velocity component v as

$$ϕ = \frac{π}{venc} v .$$

If v is out of the dynamic range (−venc, venc), phase wrapping occurs as ψ differs from ϕ by a multiple of 2π. The objective of phase unwrapping is to find ϕ based on the acquired ψ so that the underlying velocity can be properly determined.

To unwrap the phase field, one approach is to integrate the phase variation estimated as:

$$= ( Δψ ) ,$$

(hereinafter “Equation 2”) where Δψ is the spatial or temporal variation of the acquired (wrapped) phase, [symbol] is the estimated variation for the unwrapped phase by wrapping Av as in Equation 1. Equation 2 assumes that the phase variation between neighboring voxels is within the range of (−π, π), which is generally valid since the blood velocity varies continuously across the field. The phase variation integration can be treated as an optimization process and solved in a least-squares sense. This approach has been tested with 2D synthetic phase images, and the robustness can be improved by assigning proper weights to the objective function. The weighted least-squares (WLS) method has been demonstrated to improve the pressure integration with the weights generated based on the accuracy of pressure variation. A similar WLS approach can be developed and applied to the phase unwrapping of 4D flow MRI. Moreover, the divergence-free constraint can be incorporated into the WLS minimization to further improve the accuracy of the unwrapping and denoise the phase field.

### III. Exemplary Methods of Phase Unwrapping with Flow-Physics Constrained Weighted Least-Squares (CWLS)

### A. Overview

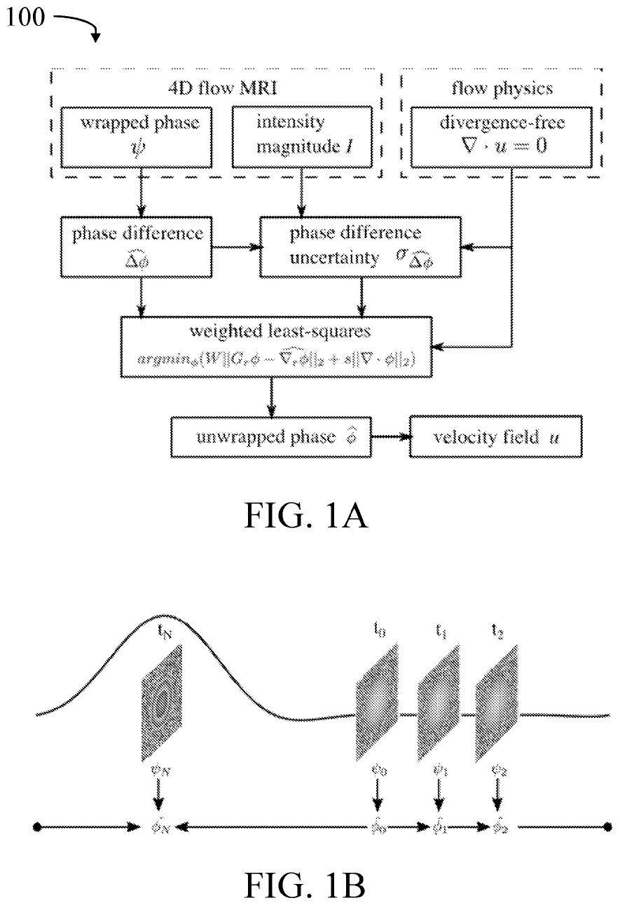

One exemplary method 100 of phase unwrapping with CWLS is presented in FIG. 1A. First, the phase variation Δψ may be calculated from the wrapped phase field v. Specifically, the spatial phase variation may be the difference between neighboring voxels, and the temporal phase variation was the difference between consecutive cardiac frames. Then [symbol] may be estimated using Equation 1. The phase gradient may be calculated as the phase variation divided by the corresponding spatial or temporal resolution, for example,

$$= / Δ r ,$$

(hereinafter “Equation 3”) where [symbol] is the spatial phase gradient, [symbol] is the spatial phase variation, and Δr is the voxel size. The subscript r represents the spatial dimension. The unwrapped phase \hat{ϕ} is spatially related to the phase gradient [symbol] as:

$${D}_{r} \hat{ϕ} = ,$$

(hereinafter “Equation 4”) where Dᵣ is the discrete spatial gradient operator consisting of Dₓ, D_{y}, and D_{z}. In addition, the divergence-free constraint reveals the following relationship between the phases of u, v, and w velocity components (denoted as ϕᵤ, ϕᵥ, and ϕ_{w}) as:

$$∇ \cdot \vec{u} ≡ {D}_{x} u + {D}_{y} v + {D}_{z} w = \frac{{venc}_{u}}{π} {D}_{x} {ϕ}_{u} + \frac{{venc}_{v}}{π} {D}_{y} {ϕ}_{v} + \frac{{venc}_{w}}{π} {D}_{z} {ϕ}_{w} = 0 ,$$

(hereinafter “Equation 5”) where ∇· represents the discrete divergence operator, [symbol] is the velocity vector containing three components as [symbol] =[u, v, w]^{T}, vencᵤ, vencᵥ, and venc_{w} are the vencs used for measuring the three velocity components u, v, and w, Dₓ, D_{y}, and D_{z} are the discrete gradient operators constructed as matrices, and ϕᵤ, ϕᵥ, and ϕ_{w} are the vectors of phases for the three velocity components. Equation 4 and Equation 5 formulate a minimization problem which can be solved using weighted least-squares as:

$$\hat{ϕ} = arg {min}_{ϕ} ( {‖ W ( {D}_{r} ϕ - ) ‖}_{2}^{2} + s {‖ \frac{{venc}_{u}}{π} {D}_{x} {ϕ}_{u} + \frac{{venc}_{v}}{π} {D}_{y} {ϕ}_{v} + \frac{{venc}_{w}}{π} {D}_{z} {ϕ}_{w} ‖}_{2}^{2} ) ,$$

(hereinafter “Equation 6”) with

$$W = diag ( \frac{1}{} ) ,$$

(hereinafter “Equation 7”) where ∥ ∥₂ represents the L2 norm, Dᵣ is the combined discrete gradient operator constructed by vertically stacking Dₓ, D_{y}, and D_{z}, ϕ is the vector consisting of ϕᵤ, ϕᵥ, and ϕ_{w}, [symbol] is the vector of the spatial phase gradients determined using Equation 3, W is the weight matrix generated based on the uncertainty of the phase gradient [symbol] , diag( ) generates the diagonal matrix with the given diagonal elements, and s is the constant controlling the level of regularization by the divergence-free constraint. The term ∥W(Dᵣϕ− [symbol] )∥₂ is the weighted residual of phase variations, and the term

$${‖ \frac{{venc}_{u}}{π} {D}_{x} {ϕ}_{u} + \frac{{venc}_{v}}{π} {D}_{y} {ϕ}_{v} + \frac{{venc}_{w}}{π} {D}_{z} {ϕ}_{w} ‖}_{2}$$

is the velocity divergence. The divergence-free constraint may be more reliable than the phase gradients since the divergence-free constraint is based on the flow-physics while the phase gradients were estimated from the measurement containing noise and errors. In order to minimize the velocity divergence, s was assigned to be significantly larger than the mean of the phase gradient weights (W̅). The residual divergence in the resulting velocity fields can be eliminated by using an s value greater than 10⁴W̅, thus s was set to 10⁶W̅ unless specified otherwise. LSQR, an iterative algorithm for sparse least-squares problems, may be employed to obtain the solution from Equation 6. The discrete gradient and divergence operators can then be constructed using the second order central (SOC) difference scheme.

### B. Field-of-View Division

To properly apply the divergence-free constraint, the field-of-view (FOV) can be divided into regions, such as three regions, denoted as the region of blood flow ( [symbol] _{ROI}), the reference points ( [symbol] _{ref}), and the rest of the FOV. The divergence minimization in Equation 6 was only applied to the voxels within [symbol] _{ROI} since the divergence-free constraint might be invalid outside the flow. The [symbol] _{ref} is defined as a layer of voxels surrounding the [symbol] _{ROI}, and was obtained by performing one iteration of morphological dilation of [symbol] _{ROI} then subtracting [symbol] _{ROI} from the dilated region. [symbol] _{ref} located in the tissue adjacent to the blood flow, which can be dynamic or static depending on the imaging location. The phase values in [symbol] _{ref} can be set to, for example, zeros prior to the unwrapping for noise elimination, and used as the boundary condition for the CWLS phase unwrapping via gradient integration. The term ∥W(Dᵣϕ− [symbol] )∥₂ in Equation 6 can be minimized in the combined region [symbol] _{ROI}∩ [symbol] _{ref}. The phase unwrapping via gradient integration was first performed with an arbitrary point set to zero. Then the median of \hat{ϕ} in [symbol] _{ref} was evaluated and subtracted from the \hat{ϕ} in the whole field in order to enforce a zero median of \hat{ϕ} in [symbol] _{ref} in order to be consistent with the boundary condition and ensure the robustness since the median is not affected by the extreme values obtained in [symbol] _{ref} due to noise. The rest of the FOV was excluded from the CWLS unwrapping to save computational effort.

### C. Uncertainty Estimation of Phase Variation

The uncertainty [symbol] of each [symbol] value needed for generating the weight matrix W in Equation 7. [symbol] was estimated as the standard deviation of the distribution of the phase variation error [symbol] ≡ [symbol] −∇ᵣϕ, where ∇ᵣϕ is the true phase gradient. [symbol] can be decomposed into two components:

$$= + ,$$

(hereinafter “Equation 8”) where [symbol] is the error component due to the measurement noise in ψ, and

$$≡ / Δ r$$

is caused by the incorrect phase variation estimation by Equation 2. Since the two error components in Equation 8 are uncorrelated, the uncertainty [symbol] can be determined as

$$= \sqrt{{( )}^{2} + {( )}^{2}} ,$$

(hereinafter “Equation 9”) where [symbol] and [symbol] are the uncertainties of [symbol] and [symbol] , respectively.

The magnitude of [symbol] can be inferred from the integration of [symbol] along closed loops in space. The smallest possible loops are 2×2 voxel rectangular loops denoted as loop elements. The integration ( [symbol] ) of each loop element equals the sum of the four [symbol] values on the loop element. Since each [symbol] value can be on multiple loop elements, the phase variation uncertainty [symbol] was approximated as the sum of

$$\frac{1}{4} \vert ∮ \vert$$

from all the loop elements. The [symbol] was calculated for all possible 2×2 voxel loop elements in the 3D field, and the value of [symbol] was additively updated. The phase gradient uncertainty [symbol] was the determined as

[symbol]

The uncertainty [symbol] for the noise component was estimated based on the spurious divergence of the velocity field as well as the intensity magnitude field I. First, the velocity-divergence field ∇·u was calculated from [symbol] using Equation 5. According to the divergence-free constraint, ∇·u is related to the phase noise

$${ϵ}_{\hat{ϕ}}^{N}$$

as:

$$\frac{{venc}_{u}}{π} {D}_{x} + \frac{{venc}_{v}}{π} {D}_{y} + \frac{{venc}_{w}}{π} {D}_{z} = ∇ \cdot \vec{u}$$

(hereinafter “Equation 10”). Similar to the velocity error estimation from velocity divergence,

$${ϵ}_{\hat{ϕ}}^{N}$$

was obtained by solving Equation 10 in a least-squares sense. The

$${ϵ}_{\hat{ϕ}}^{N}$$

was convolved with a 3D Gaussian kernel with a width of 2Δr corresponding to the three-point stencil-size of the SOC scheme to obtain the phase uncertainty field

$${ϵ}_{\hat{ϕ}}^{N , G} .$$

In addition, the root-mean-square (RMS) of

$${ϵ}_{\hat{ϕ}}^{N}$$

in [symbol] _{ROI} was calculated to represent the global phase noise level as

$${\overline{σ}}_{\hat{ϕ}}^{N} .$$

Since the noise in the phase is inversely proportional to the intensity magnitude, the ratio between the local and global phase noise uncertainty equals the reciprocal of the ratio between the local and global intensity. Thus, the phase noise uncertainty can be estimated based on the intensity field and the global phase noise uncertainty

$${\overline{σ}}_{\hat{ϕ}}^{N}$$

as:

$${\overline{σ}}_{\hat{ϕ}}^{N , I} = {\overline{σ}}_{\hat{ϕ}}^{N} \overline{I} / I ,$$

(hereinafter “Equation 11”) where Ī is the average of the intensity magnitudes in [symbol] _{ROI}. The quadratic mean of the two estimations of phase noise uncertainty was calculated as:

$${σ}_{\hat{ϕ}}^{N} = \sqrt{\frac{1}{2} [ {( {σ}_{\hat{ϕ}}^{N , G} )}^{2} + {( {σ}_{\hat{ϕ}}^{N , I} )}^{2} ]} ,$$

(hereinafter “Equation 12”) which was then propagated through the calculations of phase variation and phase gradient to acquire the phase gradient uncertainty

[symbol]

### D. Sequential Frame Unwrapping

Based on the temporal continuity of the velocity field, an unwrapped frame can be used to infer the temporally neighboring frames as:

$${\hat{ϕ}}_{i \pm 1}^{t} = {\hat{ϕ}}_{i} + 𝒲 ( {Δ}_{t} ψ ) , with {Δ}_{t} ψ = {ψ}_{i \pm 1} - {ψ}_{i} ,$$

(hereinafter “Equation 13”) where \hat{ϕ}ᵢ is the unwrapped phase at i^{th} cardiac frame, Δₜψ is the temporal phase variation, and

$${\hat{ϕ}}_{i \pm 1}^{t}$$

is the temporally unwrapped phase at the neighboring frames i±1. The temporally unwrapped phase \hat{ϕ}^{t} was utilized in the CWLS unwrapping. First, the spatial variation of \hat{ϕ}^{t} was combined with the estimation from Equation 2 to obtain the spatial phase variation as:

$$= \frac{1}{2} ( {Δ}_{r} {\hat{ϕ}}^{t} + 𝒲 ( {Δ}_{r} ψ ) ) ,$$

(hereinafter “Equation 14”) which was employed in the phase variation integration by Equation 6. Second, the deviation between Δᵣ\hat{ϕ}^{t} and [symbol] (Δᵣψ) was used to update the phase gradient uncertainty as:

$$= \sqrt{{( )}^{2} + {( )}^{2} + {( \frac{{Δ}_{r} {\hat{ϕ}}^{t} - 𝒲 ( {Δ}_{r} ψ )}{Δ r} )}^{2}} ,$$

(hereinafter “Equation 15”) which was employed to generate the weight matrix W in Equation 7. In addition, \hat{ϕ}^{t} was used as the initial field for solving Equation 6 with the iterative LSQR algorithm.

Since the reliability of

$${\hat{ϕ}}_{i \pm 1}^{t}$$

depends on the accuracy of \hat{ϕ}ᵢ, it is preferable to perform the temporal phase unwrapping from a less-wrapped frame towards a more-wrapped one. The frame sequences were adopted to start from the frame with lowest average velocity magnitude towards the frame with highest average velocity magnitude along both the forward and backward temporal directions as demonstrated in FIG. 1B. FIG. 1B shows a sequence of temporal phase unwrapping from the time point with lowest average velocity (at t₀) to the time point with highest average velocity (at t_{N}) along the forward and backward directions in a cyclic manner, with the waveform demonstrating the flow rate in one cardiac cycle. Particularly, the frame with highest flow rate was unwrapped twice with the two temporal sequences as both neighboring timeframes had lower flow rates. Each of the two unwrapping operations on the frame with highest flow rate was performed independently and initialized with one of the neighboring frames, yielding two unwrapping results which were similar in general. The average of the two unwrapped fields were taken as the final result since taking the average can reduce the uncertainty compared to a single sample. The proposed temporal sequences can prevent the propagation of unwrapping errors from severely wrapped frames to the less-wrapped ones. The starting and the ending timepoints can be approximated as the peak diastole or peak systole depending on the locations in the cardiovascular system.

### E. Synthetic Phase Data Generation

To evaluate the performance of the CWLS method, synthetic phase data was generated from computational fluid dynamics (CFD) simulated LV flow velocity fields. The CFD results were obtained on unstructured computational mesh with 180,000 tetrahedral cells and linearly interpolated to a fine Cartesian grid with spatial resolution of 0.2 mm. Complex-valued signal was generated at each grid node based on each velocity component as:

$${M}_{fine} = I exp ( \frac{i π u}{venc} ) ,$$

(hereinafter “Equation 16”) where M_{fine} denotes the complex signal at the fine grid node, I is the signal magnitude, and u is the velocity component at grid node. Another Cartesian grid with a resolution of 2.5 mm was employed as the MRI grid ( [symbol] _{MRI}) with each grid point corresponding to a voxel-center, in order to be consistent with the typical resolutions of heart scans. The complex-valued signal at each voxel-center of the synthetic 4D flow MRI data was generated by convolving the signal on the fine Cartesian grid with a sinc-function kernel (K) as:

$$K ( x , y , z ) = sin c ( \frac{x}{Δ x} ) \times sin c ( \frac{y}{Δ y} ) \times sin c ( \frac{z}{Δ z} ) ,$$

(hereinafter “Equation 17”) with

$$sin c ( x ) = \frac{sin ( π x )}{π x} ,$$

where Δx, Δy, and Δz represent the spatial resolution of the MRI grid. Previous studies have shown that the spatial blurring of Cartesian 4D flow MRI measurement due to limited coverage of the k-space equals to the convolution with the sinc-function kernel, and convolving with the sinc-function kernel has been used to simulate 4D flow MRI acquisitions. One reference (M₀) and three flow-sensitive datasets (Mᵤ, Mᵥ, and M_{w}) were simulated following a four-point reference method. Each flow-sensitive dataset was created based on the field of a velocity component, and the reference dataset was generated from a zero-phase field such that the phase difference between the flow-sensitive and the reference datasets was consistent with the velocity field as in real applications. The signal noise ϵ in each component of the complex-valued data was assumed to be normally distributed with a standard deviation of σ_{I}=Ī/SNR_{I}, where SNR is the intensity magnitude based SNR. The wrapped phase data ψ for each velocity component was generated from the complex-valued data, e.g.:

$${ψ}_{u} = angle ( {M}_{u} * {M}_{0}^{*} ) ,$$

(hereinafter “Equation 18”) where ψᵤ is the phase for u velocity component, M₀* is the complex conjugate of M₀, and angle( ) means calculating the angle from a complex signal as:

$$angle ( a + bi ) = arctan ( \frac{b}{a} )$$

(hereinafter “Equation 19”).

Since the reference dataset was shared among the three flow-sensitive datasets, the phase noise of different velocity components were correlated in a similar way as the real phase data. The intensity magnitude field/was allowed to vary spatially as commonly seen from the FOV of 4D flow MRI. The spatial distribution of/was defined as:

$$I = 1. - 0.5 ( \frac{x}{{L}_{domain}} ) ,$$

(hereinafter “Equation 20”) where L_{domain} is the total length of the FOV along the x direction. The I outside D_{ROI} was multiplied with 0.2 to mimic the low intensity outside the lumen. In addition to the predefined bulk variation, I would also vary locally due to the noise and the intravoxel dephasing effect caused by the spatiotemporal variation of velocity.

Since the SNR of MRI acquisitions can be greater than 100 for in vitro measurements and less than 10 for in vivo measurements, the following six values were employed to represent a wide range of SNRI as: 100, 50, 20, 10, 5, and 2. A wide range of vencs were also employed to test CWLS on different levels of phase wrapping. The venc ratio (VR) defined as the ratio between the venc and the maximum flow velocity was varied from 0.1 to 0.9 in increments of 0.1. In total, 54 test cases were created with different combinations of SNRI and VR.

To determine the effect of spatial resolution on CWLS unwrapping, several additional datasets were created using the same approach with MRI grid resolution varying from 2 to 6 mm in increments of 1 mm. For each spatial resolution, 10 datasets were created with an SNR of 10 and VR from 0.1 to 1.0 in increments of 0.1.

The mask of [symbol] _{ROI} was generated for each dataset and each time frame based on the geometry available from the CFD simulation. A voxel was considered to be in the blood flow domain if the voxel-center was within the geometry at the time instant.

### F. In Vitro 4D Poiseuille Flow Measurement

Steady, laminar Poiseuille flow in a circular pipe was measured using 4D flow MRI with different vencs. The working fluid was a blood mimicking water-glycerol (60:40 by volume) solution with a density of 1110 kg/m³ and viscosity of 0.00372 Pa·s. A small amount (0.66 mg/mL) of Gadolinium contrast was added to enhance the SNR of the scan without altering the rheology of the fluid. A computer-controlled gear pump was used to drive the working fluid at a steady flow rate of 7.6 mL/s. The diameter of the pipe was 12.7 mm, and the length was sufficiently long prior to entering the FOV such that the velocity profile was fully developed. Three dual-venc (DV) acquisitions (denoted as A, B, and C) were performed on a Siemens 3T PRISMA scanner with a spatial resolution of 0.85×0.85×0.8 mm³. The dual-venc acquisitions were split up, and the low and high venc acquisitions were analyzed separately, thus yielding 6 datasets with vencs ranging from 4 to 16 cm/s as presented in Table 1 below.

The expected maximum velocity in the field was 12 cm/s. Each dataset contained 12 time frames with a temporal resolution of 120.4 ms. The echo time (TE) and repetition time (TR) are presented in Table 1. The bandwidth was 455 kHz and flip angle was 15°. The mask of [symbol] _{ROI} was generated based on the position and radius of the pipe. A voxel was considered to be within the flow if the distance from its center to the centerline of the pipe was less than the pipe radius. The SNR_{I} values were calculated for each acquisition as SNR_{I}=Ī/σ_{I}, where or is the standard deviation of I across the 12 frames, and Ī is the average of/within [symbol] _{ROI}. The SNR_{I} values are given in Table 1.

### G. In Vivo Aortic 4D Flow MRI Measurement

In vivo aortic 4D flow MRI data was used to evaluate the performance of CWLS. Aortic flow was measured from 12 patients with bicuspid aortic valve (BAV), 12 patients with tricuspid aortic valve and aortic aneurysm (TAV-AA), and six healthy control subjects with tricuspid aortic valve. The scans were performed in a sagittal oblique volume on a 1.5 T scanner (MAGNETOM Avanto, Aera, Siemens, Erlangen, Germany) with prospective ECG gating and during free-breathing. All patients (BAV and TAV-AA) except the control subjects were imaged with gadolinium-based contrast (Magnevist, Ablavar, or Gadavist). The voxel sizes were 2-2.5 mm isotropic in-plane with a slice thickness of 2.4-3.2 mm. The temporal resolution was 37.6-39.2 ms with 10-25 cardiac time frames. TE/TR were 2.184-2.463 ms/4.6-4.9 ms, flip angle was 7° in controls and 15° in patients, and the bandwidth was 446-460 kHz. A single venc was used for each scan. The venc was 150-350 cm/s for BAV patients, 150-200 cm/s for TAV-AA patients, and 150 cm/s for control subjects. All patient data for this HIPPA compliant and IRB approved study were retrospectively included with waiver of consent. The mask of [symbol] _{ROI} for each dataset was approximated by thresholding the time-averaged product of the intensity and the magnitude of the phase components

$$( I \cdot \sqrt{{ψ}_{u}^{2} + {ψ}_{v}^{2} + {ψ}_{w}^{2}} )$$

[49] and manually corrected by an expert observer using Mimics.

In vivo datasets were assessed for aliasing, with four TAV-AA and four BAV datasets containing velocity aliasing, while no velocity aliasing was observed in the remaining 22 data sets. Phase unwrapping was applied to the data sets with velocity aliasing, and the resulting velocity fields were analyzed to assess the performance. For datasets without aliasing, the phase data were artificially wrapped based on virtual vencs that were lower than the vencs from original scans as

$$𝒲 ( \frac{π V}{venc} ) ,$$

where V is the original velocity data and venc is the virtual venc. This wrapping operation maintains the mathematical relationship between wrapped and unwrapped phase data without bringing additional noise or error to the phase field. Five VRs ranging from 0.1 to 0.5 were employed to set the virtual vencs based on the maximum velocity value within the blood flow. Outliers were excluded from the maximum velocity calculation using universal outlier detection (UOD) followed by median filtering on the unaliased velocity data. The originally unaliased datasets were used as the benchmark to assess unwrapping performance. Since the measurement noise in the benchmark datasets could affect the error analysis on the unwrapped phase fields, UOD was applied to the benchmark phase field to remove outliers.

### H. Performance Evaluation

The performance of CWLS on phase unwrapping and denoising was assessed by analyzing the unwrapped phase field as well as the resulting velocity field obtained by multiplying the unwrapped phase by venc/π. The current state-of-the-art 4D Lap was also employed in this study and compared to CWLS. 4D Lap unwraps time-resolved phase data along temporal dimension and all three spatial dimensions by evaluating the phase Laplacian with Fourier transform. All of the preprocessing was kept constant between CWLS and 4D Lap such that the input phase data were same between the unwrapping techniques.

To assess the overall performance on each test case for the synthetic phase data of LV flow, the unwrapped phase \hat{ϕ} was compared to the true phase ϕ generated from CFD results voxel by voxel at each cardiac frame. A voxel was considered as wrapped if the deviation |\hat{ϕ}−ϕ| was greater than π. The success rate (SR) of phase unwrapping was calculated as:

$$SR = 1 - \frac{{N}_{𝒲 , \hat{ϕ}}}{{N}_{𝒲 , ψ}} ,$$

(hereinafter “Equation 21”) where N [symbol] _{,\hat{ϕ}} is the total number of wrapped voxels in the unwrapped data, and N [symbol] _{,ψ} is the total number of wrapped voxels in the synthetic data. N [symbol] _{,\hat{ϕ}} and N [symbol] _{,ψ} were counted within D_{ROI} for each of the 3 velocity components at each frame, which were then summed together as

$${N}_{𝒲 , \hat{ϕ}} = {\sum }_{i = 1}^{N} ( {N}_{𝒲 ,}^{i} + {N}_{𝒲 ,}^{i} + {N}_{𝒲 ,}^{i} ) ,$$

where the superscript i indicates the i^{th} cardiac frame. SR=1 means that all voxels were correctly unwrapped. The SR can be less than 0 if the unwrapping created more wrapped voxels than the original data. The error in the resulting velocity (ϵ_{V}) was calculated as the deviation from the CFD results. To evaluate the accuracy of the resulting velocity fields, the velocity error level (Vₑᵣᵣₒᵣ) was calculated as:

$${V}_{error} = \frac{RMS ( {ϵ}_{V} )}{\overline{\vert V \vert}} \times 100 \% ,$$

(hereinafter “Equation 22”) where |̅V̅|̅ is the average velocity magnitude in D_{ROI}, and RMS(ϵ_{V}) represents the RMS velocity error in D_{ROI}.

For the in vitro 4D Poiseuille flow, the unwrapped phase \hat{ϕ} data was compared with the true phase ϕ generated from the analytical velocity fields described by:

$${u}_{r} = 0 , {u}_{θ} = 0 , {u}_{z} ( r ) = \frac{2 Q}{π {R}^{4}} ( {R}^{2} - {r}^{2} ) ,$$

(hereinafter “Equation 23”) where uᵣ is the radial velocity component, u_{θ} is the circumferential velocity component, u_{z} is the axial (along z-axis) velocity component (m/s), r is the radial distance from the pipe centerline (m), R is the pipe radius (m), and Q is the volumetric flow rate (m³/s). The number of wrapped voxels N [symbol] _{,ψ} and N [symbol] _{,\hat{ϕ}} were calculated from the acquired phase fields and the unwrapped phase fields, respectively. To quantify the noise level, the VNRs were determined from the resulting velocity fields as:

$$VNR = \frac{\overline{\vert V \vert}}{RMS ( {σ}_{V} )} ,$$

(hereinafter “Equation 24”) where σ_{V} is the velocity standard deviation across 12 frames, and RMS(σ_{V}) is the RMS of all the σ_{V} within D_{ROI}. The wrapped voxels were excluded from the VNR calculation such that the VNR only represented the noise level. From the unwrapped velocity fields using CWLS and 4D Lap, the WSS was calculated from the velocity gradients determined using thin-plate spline radial basis function interpolation with the non-slip (zero velocity) boundary condition applied on the wall. The WSS error (ϵ_{WSS}) was determined by comparing the magnitude of the WSS vector to the analytical value determined as:

$$\vert WSS \vert = \frac{4 μ Q}{π {R}^{3}} ,$$

(hereinafter “Equation 25”) where μ is the dynamic viscosity of the fluid (Pa·s). For each dataset, the relative ϵ_{WSS} was calculated as the RMS of ϵ_{WSS} in D_{ROI} normalized by the analytical WSS magnitude.

To evaluate the performance with the in vivo aortic 4D flow data, the SRs defined by Equation 21 on the artificially wrapped datasets were determined by comparing the unwrapped phase to the benchmark (the originally unaliased datasets). Because benchmark data is not available for the eight datasets with real aliasing, the error in the resulting velocity fields were estimated based on the velocity divergence using the least-squares algorithm, which was then employed to calculate the V_{errorS} using (Equation 22). To indicate the level of wrapping in the original phase data, the venc ratio was estimated based on the average of the maximum velocity values from the CWLS and 4D Lap unwrapped fields.

### IV. Selected Experimental Results from Performance of Exemplary Methods Described Herein

### A. Synthetic Phase Data of LV Flow

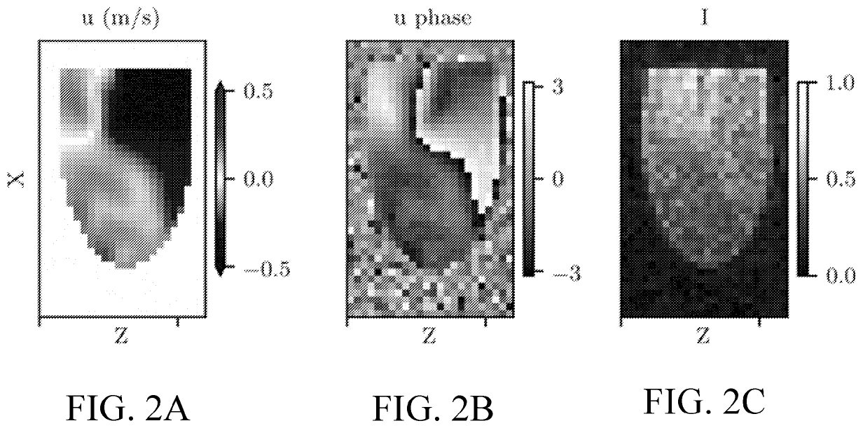

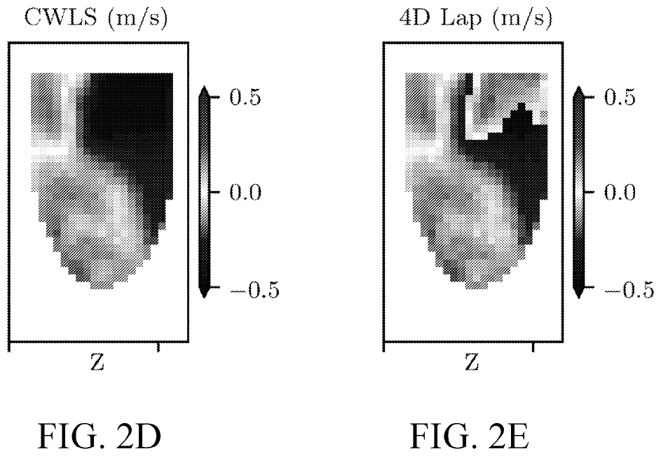

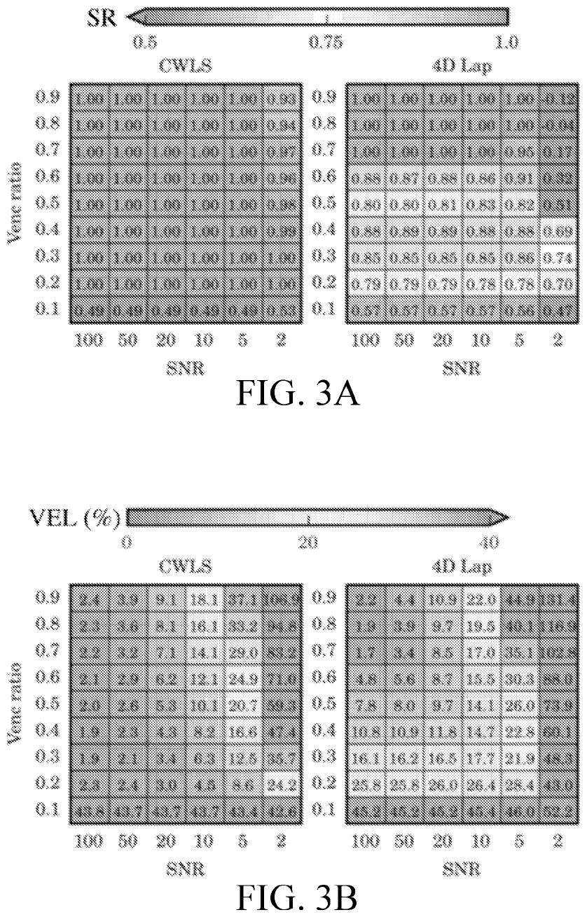

The u velocity field at peak diastole on the MRI grid is shown in FIG. 2A. The generated phase and magnitude intensity fields for VR=0.2 and SNRI=10 are shown in FIGS. 2B and 2C, respectively. The unwrapped u-component velocity fields at peak diastole are compared in FIGS. 2D and 2E, respectively, for the case of SNRI=10 and VR=0.2. With 4D Lap, the large region of wrapped voxels in inflow jet remained, while all voxels were correctly unwrapped by CWLS. The SRs and Vₑᵣᵣₒᵣₛ of all the cases are compared in FIG. 3A between CWLS and 4D Lap. The CWLS completely unwrapped the phase data for most cases with VR≥0.2 and SNR_{I}≥5. Even with significant amount of noise (SNR_{I}=2), the SRs were consistently greater than 0.8. Compared to 4D Lap, CWLS was more robust to noise and more reliable for low-venc acquisitions. The CWLS method effectively reduced the Vₑᵣᵣₒᵣ in most cases compared to 4D Lap. The improvement was significant for the cases where 4D Lap failed to unwrap all the voxels and led to Vₑᵣᵣₒᵣ reduction as much as 500%. It is also worth noting that CWLS reduced the Vₑᵣᵣₒᵣₛ by around 20% compared the 4D Lap results for the low-SNRI cases where both methods completely unwrapped the phase.

The effects of spatial resolution on the performances of CWLS and 4D Lap were presented in FIG. 3B in terms of the SRs and Vₑᵣᵣₒᵣₛ from the datasets with an SNR of 10, VR from 0.1 to 1.0, and grid size from 2 to 6 mm. The SR by CWLS remained around 1.0 for all the cases with VR>0.1, whereas the SR by 4D Lap decreased with the increase of grid size for cases for VR from 0.2 to 0.4. Thus, greater improvement was achieved by CWLS compared to 4D Lap for cases with larger voxel size. The Vₑᵣᵣₒᵣₛ by CWLS were consistently lower than 4D Lap for all the cases with VR>0.1. At each VR, the Vₑᵣᵣₒᵣ by CWLS slightly increased with the increase of grid size due to the voxel-averaging effect.

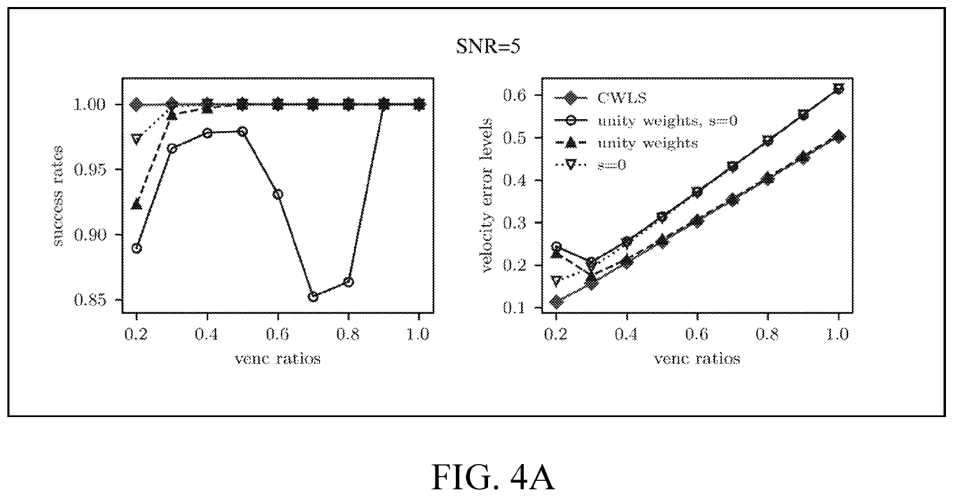

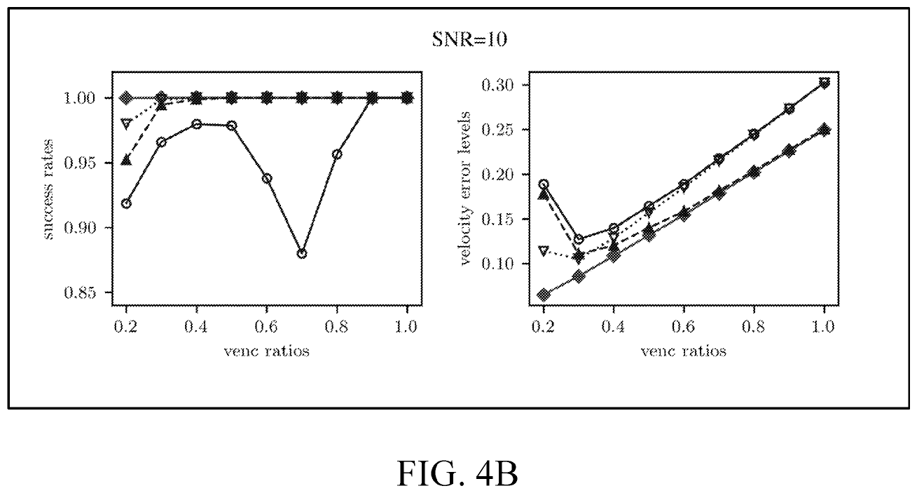

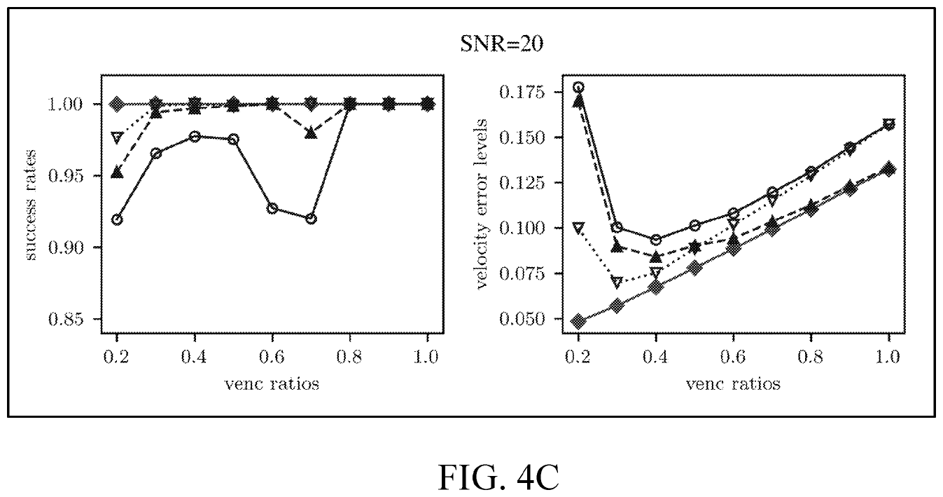

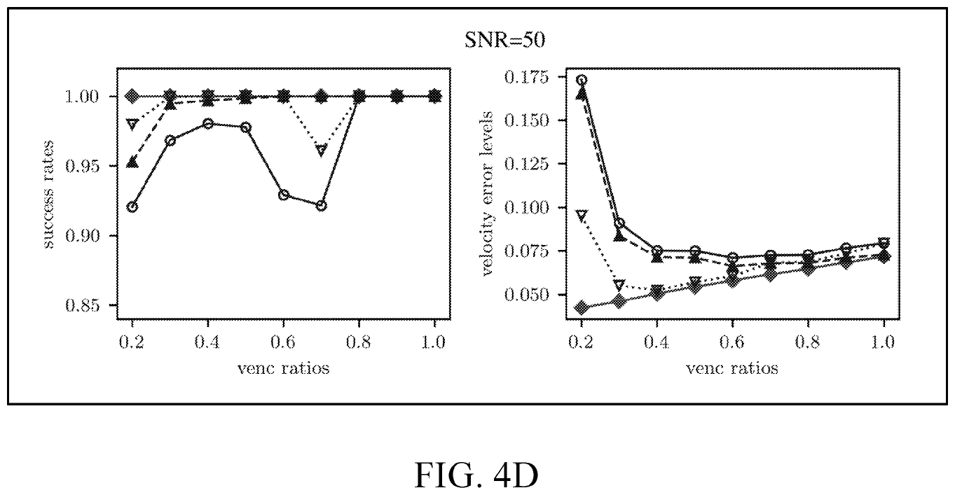

The effect of the uncertainty-based weighting and the divergence-free regularization was demonstrated by comparing CWLS with the unwrapping frameworks with unity weights or zero regularization constant s. With a SNR of 10 and VR from 0.2 to 1.0, the SRs and Vₑᵣᵣₒᵣₛ of the different unwrapping frameworks are presented in FIG. 4 as functions of VR. The method of “unity weights” means applying unity weights while “s=0” means setting s to zero, and “unity weights, s=0” employed both unity weights and zero regularization constant. As shown in FIG. 4, CWLS yielded a SR around 1.0 for all the cases. Without either the uncertainty-based weighting or the divergence-free regularization, the SRs were affected for cases with VR<0.4, indicating that both operations improved the unwrapping results at low VR. The “unity weights, s=0” yielded the lowest SRs for all cases with VR<0.8. For the cases with VR≥0.8, the phase data were unwrapped completely by all the methods as SR=1.0, and the Vₑᵣᵣₒᵣₛ of the two methods with divergence-free regularization were lower than the other two, indicating the denoising effect of the divergence-free regularization.

### B. In Vitro 4D Poiseuille Flow

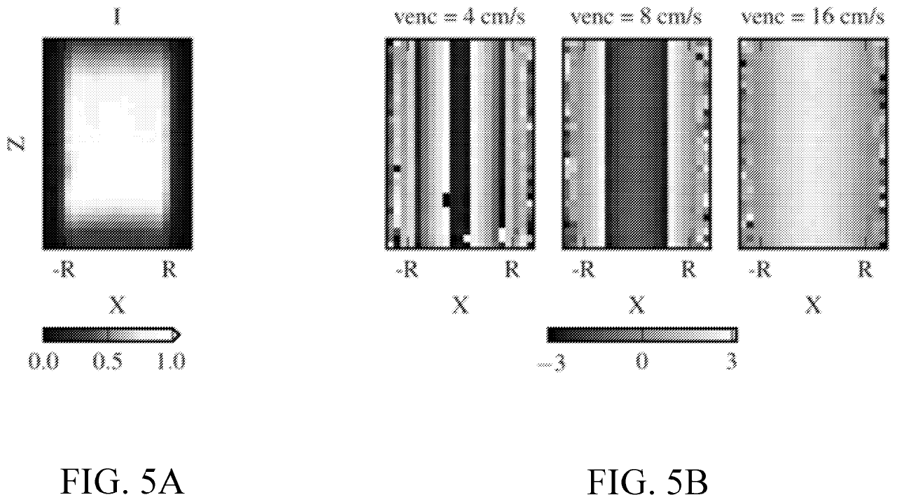

For the Poiseuille flow, the analytical solution had a maximum axial velocity (wₘₐₓ) of 12 cm/s at centerline. The VRs of the six acquisitions were determined accordingly and given in Table 1. The intensity magnitude and phase fields from three datasets are presented in FIGS. 5A and 5B. The intensity magnitude was higher near the center of the FOV, while it was lower near the pipe wall (partial volume effect) and on the edges of the FOV. The voxels along the centerline of the phase field were wrapped twice at venc=4 cm/s and were wrapped once at venc=8 cm/s.

The unwrapped phase \hat{ϕ} data was compared with the true phase ϕ generated from the analytical velocity fields. The number of wrapped voxels N [symbol] _{,ψ} and N [symbol] _{,\hat{ϕ}} are presented in Table 1. As a reference, the total number of voxels within D_{ROI} (N_{ROI}) was 63720. One aliased voxel existed in the dataset with venc=16 cm/s, which was due to measurement noise. The 4D Lap unwrapped most of the voxels and failed to unwrap 2 to 104 wrapped voxels for each dataset, while CWLS completely unwrapped five datasets and failed to unwrap only one wrapped voxel for venc=4 cm/s. With venc=16 cm/s, the 4D Lap created nine more wrapped voxels compared to the unprocessed data. The VNRs of the resulting velocity fields are presented in Table 1 together with the percentage increase of VNR by CWLS compared to 4D Lap. Compared to 4D Lap, the CWLS VNRs were 39.1-60.6% higher, demonstrating the denoising effect by CWLS unwrapping on velocity accuracy. With CWLS, the VNR was 131% higher using a venc of 4 cm/s than the VNR at a venc of 16 cm/s.

TABLE 1

| DV Acquisitions | | A | B | C | A | B | C |
|---|---|---|---|---|---|---|---|
| Venc (cm/s) | | 4 | 6 | 8 | 8 | 12 | 16 |
| TE (ms) | | 7.47 | 6.47 | 5.87 | 7.47 | 6.47 | 5.87 |
| TR (ms) | | 10.2 | 9.2 | 8.6 | 10.2 | 9.2 | 8.6 |
| SNR_{I} | | 60.9 | 54.1 | 47.9 | 60.9 | 54.1 | 47.9 |
| N_{W} | | 41919 | 32819 | 24128 | 23434 | 3925 | 1 |
| N_{W} | CWLS | 1 | 0 | 0 | 0 | 0 | 0 |
| | 4D Lap | 104 | 2 | 20 | 38 | 4 | 10 |
| VNR | CWLS | 33.2 | 38.4 | 28.1 | 16 | 18.5 | 14.4 |
| | 4D Lap | 23.7 | 26.4 | 17.5 | 10.7 | 13.3 | 9.4 |
| VNR Improvement (%) | | 40 | 46 | 61 | 50 | 39 | 53 |
| Mean WSS (Pa) | CWLS | 0.17 | 0.16 | 0.17 | 0.2 | 0.18 | 0.2 |
| | 4D Lap | 0.2 | 0.17 | 0.19 | 0.23 | 0.2 | 0.22 |
| Relative ϵ_{WSS} | CWLS | 0.45 | 0.37 | 0.42 | 0.68 | 0.51 | 0.65 |
| | 4D Lap | 0.7 | 0.56 | 0.78 | 1.38 | 1.02 | 1.5 |

| | | | | | | |
|---|---|---|---|---|---|---|
| ϵ_{WSS} Reduction (%) | 56 | 53 | 85 | 105 | 102 | 130 |

Specifically, Table 1 shows the venc, intensity-based signal-to-noise ratio (SNRI), number of wrapped voxels (N_{W}), velocity-to-noise ratio (VNR), mean WSS magnitude, and relative WSS magnitude error (ϵ_{WSS}) for the each in vitro Poiseuille flow dataset with CWLS and 4D Lap unwrapping

### C. In Vivo Aortic 4D Flow MRI

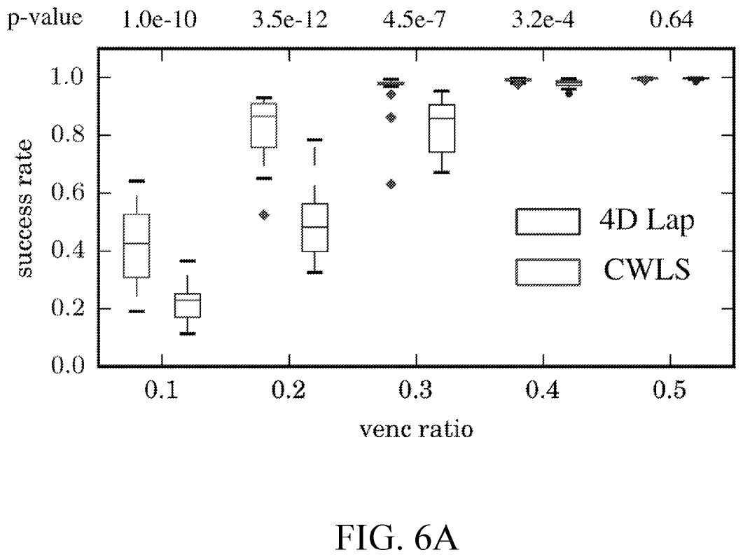

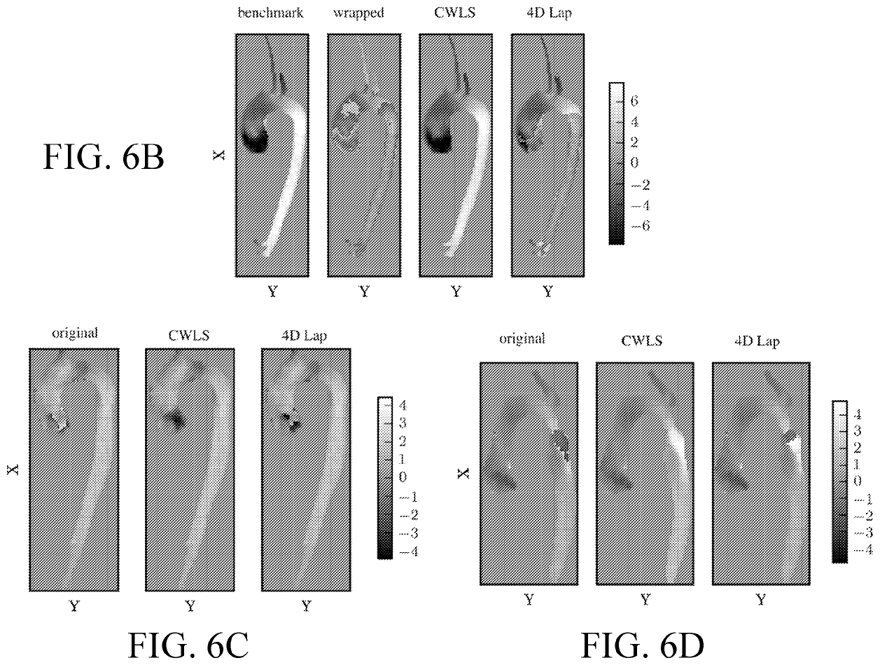

The SRs of 22 datasets for each VR are presented in FIG. 6A using boxplot. The p-values from paired sample t-test between the SRs by CWLS and 4D Lap are also reported in FIG. 6A, which indicated statistically significant difference (p-value<0.05) between the performances of the two methods at VRs of 0.1 to 0.4. The median SR is given in Table 2. Compared to 4D Lap, the improvement by CWLS was dramatic for VRs at 0.2 and 0.3. At a VR of 0.2, the median SR value was 81% higher by CWLS compared to 4D Lap. Examples of the unwrapped phase fields are given in FIG. 6B for a BAV dataset with a VR of 0.3 together with the benchmark ϕ and the wrapped phase y. Doubly-wrapped voxels can be observed in/near the aortic valve and in the descending aorta. CWLS completely unwrapped these voxels, while a large portion of wrapped voxels still remained from 4D Lap.

TABLE 2

| VR | | 0.1 | 0.2 | 0.3 | 0.4 | 0.5 |
|---|---|---|---|---|---|---|
| Median of SRs | CWLS | 0.43 | 0.87 | 0.98 | 0.99 | 1 |
| | 4D Lap | 0.23 | 0.48 | 0.86 | 0.98 | 1 |

Specifically, Table 2 shows the median success rates (SR) for each venc ratio (VR) of the artificially wrapped in vivo aortic datasets with CWLS and 4D Lap. The VRs and Vₑᵣᵣₒᵣₛ for the in vivo datasets with real velocity aliasing are given in Table 3. The Vₑᵣᵣₒᵣₛ of the 4D Lap processed fields were minimally 10 times higher than the Vₑᵣᵣₒᵣₛ of the CWLS results. The unwrapped phase fields from one BAV case and one TAV-AA case with real aliasing are presented in FIGS. 6C and 6D. With 4D Lap unwrapping, phase jumps were observed near the aortic valve for the BAV case, as well as wrapped voxels in the descending aorta for the TAV-AA case. The CWLS completely unwrapped the voxels in the displayed field. The computational costs by CWLS on the aliased in vivo datasets were quantified with respect to the number of voxels (Nᵥₒₓₑₗₛ) within D_{ROI}∪D_{ref}. As Nᵥₒₓₑₗₛ increased from 17640 to 35574, both the number of LSQR iterations and time-cost per iteration increased, resulting in a linear increase of the total time-cost per timeframe from 100 to 310 s. It should be noted that the computations were carried using a workstation with 16 cores (Intel Xeon CPU E5-2450 v2), and the time-cost may change with different computational capacity.

TABLE 3

| | VR | | 0.51 | 0.7 | 0.63 | 0.72 |
|---|---|---|---|---|---|---|
| BAV | Vₑᵣᵣₒᵣ (%) | CWLS | 2.9 | 2.6 | 2.3 | 1.9 |
| | | 4D Lap | 55.9 | 34 | 41.9 | 30.8 |

| | VR | | 0.54 | 0.64 | 0.95 | 0.71 |
|---|---|---|---|---|---|---|
| TAV-AA | Vₑᵣᵣₒᵣ (%) | CWLS | 1.7 | 2.1 | 1.7 | 2.8 |
| | | 4D Lap | 36.7 | 30.1 | 30.5 | 35.3 |

Specifically, Table 3 shows the venc ratios (VR) of the acquisitions and the velocity error levels (Vₑᵣᵣₒᵣ) of the resulting velocity fields for the eight in vivo aortic datasets with real aliasing by CWLS and 4D Lap unwrapping.

### D. Conclusions

The described method algorithmically unwraps the phase data without the need of additional high-venc acquisition. Notably, the data can be unwrapped via the method steps described an infinite number of times. In some embodiments, the data can be unwrapped from five to 10 times. The performance of CWLS method was evaluated and demonstrated with synthetic phase data, in vitro measurement of Poiseuille flow, and in vivo aortic 4D flow data. By incorporating the divergence-free constraint and using the robust WLS integration algorithm, CWLS reliably and robustly unwrapped the phase data with a venc as low as 20% of the maximum velocity and a SNR as low as five, and also reduces the phase noise. As a consequence, CWLS improved the accuracy of the obtained velocity and hemodynamic quantities.

The CWLS method allows for the use of lower venc to obtain more accurate velocity and subsequent hemodynamic quantities in clinical applications of 4D flow MRI. Overall, a VNR increase of more than 100% can be achieved by using lower-venc acquisitions and the CWLS unwrapping according to the analysis on the in vitro Poiseuille flow. In addition, the CWLS method does not require any change in the 4D flow MRI acquisition in comparison with the multi-venc approaches which need additional high-venc acquisition with a 25-75% increase in scan time. In applications where two 4D flow MRI scans are typically required for measuring venous and arterial flow with different vencs such as in the liver or brain, CWLS can reduce the scan time by omitting the high-venc acquisition and unwrapping the low-venc data.

Compared to 4D Lap, CWLS is more reliable for severely wrapped data, and more robust to noise and low spatial resolution. Unlike the 4D Lap method which unwraps along four dimensions in a single step, CWLS sequentially unwraps each time frame and employs WLS for spatial unwrapping. The time sequence proposed in section III-D prevents the error propagation from more-wrapped frames to less-wrapped frames, and the WLS integration mitigates the error propagation across the field. Moreover, CWLS incorporates the divergence-free constraint to regularize and denoise the phase field. Thus, CWLS better handles phase singularity and reduces noise during unwrapping. The advantage of 4D Lap over CWLS is its ease of use and low computational cost. Neither method needs aliasing-free reference timeframes as required by other temporal unwrapping algorithms. Compared to the unwrapping method which resolves phase singularity with branch cut surfaces, the CWLS method does not rely on the estimation of phase singularity loops, making it more scalable for large and complex datasets. The advantage of CWLS over the 4D gradient based phase unwrapping is that CWLS can unwrap voxels wrapped multiple times and large wrapped regions.

There are certain limitations of the CWLS method. First, the computational cost of CWLS was expensive compared to 4D Lap. Using a workstation with 16 cores (Intel Xeon CPU E5-2450 v2), the processing of each in vivo dataset took 1-2 hours, whereas 4D Lap completed the unwrapping within seconds. Another limitation of CWLS was that the FOV needed to be segmented prior to unwrapping, which can be difficult for acquisitions with tissue movement despite the recent development on 3D segmentation algorithms. The segmentation applied to the in vivo aortic data based on the time-averaged quantity did not consider the motion of aorta and might affect the CWLS unwrapping. However, the CWLS still showed superior performance compared to 4D Lap on the in vivo aortic data with this segmentation. It is also worth noting that the CWLS unwrapping depends on the phase variation estimated using Equation 2 with the assumption that the phase variation between neighboring voxels are within (−π, π). Using an extremely low venc can violate the assumption and therefore affect the performance of CWLS as suggested by the low SRs from the cases with VR=0.1 in FIGS. 3A and 3B.

Furthermore, there can be some limitations. First, the benchmark phase data for the eight real-aliasing in vivo datasets was unavailable to evaluate the SR of unwrapping. Instead, the velocity errors were estimated from the velocity divergence and compared the Vₑᵣᵣₒᵣₛ between results from CWLS and 4D Lap. However, it should be noted that this divergence-based error metric could underestimate the error level from CWLS which penalized the velocity divergence during phase unwrapping. In vivo dual-venc datasets can be acquired in future studies and used as benchmark to evaluate the performance of phase unwrapping on low-venc acquisitions. Moreover, further investigation on CWLS unwrapping needs to be performed for severely wrapped in vivo datasets with VRs lower than 0.5. In addition, the intra-voxel phase dispersion due to the aortic valve pathologies was not considered in the synthetic data generation or the in vitro experiment, limiting the performance evaluation of CWLS on data with this artifact.

In conclusion, this study introduces a divergence-free constrained phase unwrapping method for 4D flow MRI and evaluates its performance with synthetic phase data, in vitro measurement of Poiseuille flow, as well as in vivo aortic 4D flow data. The proposed method is reliable with severely wrapped data and robust to noise. The method also denoises the phase field and thus enhances the VNR of the resulting velocity data. The method can benefit clinical applications of 4D flow MRI as it improves the accuracy of acquired velocity and hemodynamic quantities.

### V. Exemplary Systems Configured for Phase Unwrapping with Flow-Physics Constrained Computations

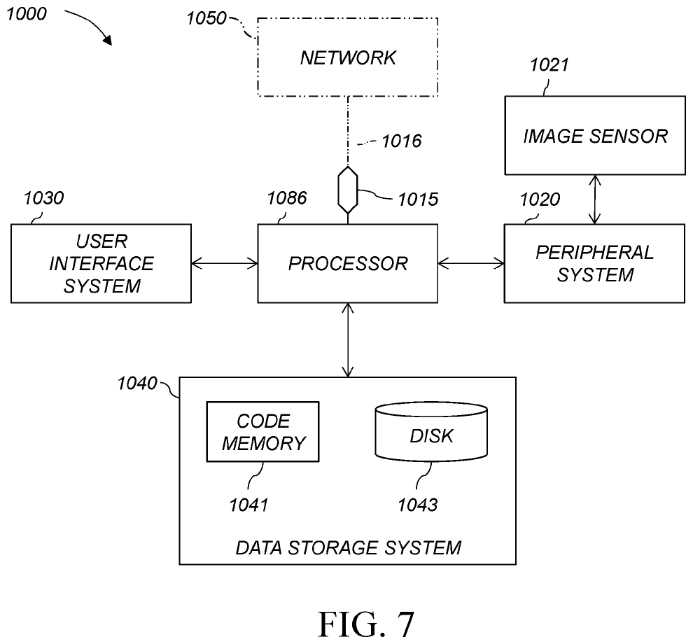

FIG. 7 is a high-level diagram showing the components of an exemplary data-processing system 1000 for analyzing data and performing the methods and analyses described herein, and related components. The system includes a processor 1086, a peripheral system 1020, a user interface system 1030, and a data storage system 1040. The peripheral system 1020, the user interface system 1030 and the data storage system 1040 are communicatively connected to the processor 1086. Processor 1086 can be communicatively connected to network 1050 (shown in phantom), e.g., the Internet or a leased line, as discussed below. The imaging data described herein to be processed using the methods described may be obtained using imaging sensors or cameras 1021 and/or displayed using display units (included in user interface system 1030) which can each include one or more of systems 1086, 1020, 1030, 1040, and can each connect to one or more network(s) 1050. Processor 1086, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), or digital signal processors (DSPs).

Processor 1086 can implement processes of various aspects described herein. Processor 1086 can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise. Processor 1086 can include Harvard-architecture components, modified-Harvard-architecture components, or Von-Neumann-architecture components.

The phrase “communicatively connected” includes any type of connection, wired or wireless, for communicating data between devices or processors. These devices or processors can be located in physical proximity or not. For example, subsystems such as peripheral system 1020, user interface system 1030, and data storage system 1040 are shown separately from the data processing system 1086 but can be stored completely or partially within the data processing system 1086.

The peripheral system 1020 can include one or more devices configured to provide digital content records to the processor 1086. For example, the peripheral system 1020 can include digital still cameras, digital video cameras, cellular phones, or other data processors. The processor 1086, upon receipt of digital content records from a device in the peripheral system 1020, can store such digital content records in the data storage system 1040.

The user interface system 1030 can include a mouse, a keyboard, another computer (connected, e.g., via a network or a null-modem cable), or any device or combination of devices from which data is input to the processor 1086. The user interface system 1030 also can include a display device (e.g., a display screen), a processor-accessible memory, or any device or combination of devices to which data is output by the processor 1086. The user interface system 1030 and the data storage system 1040 can share a processor-accessible memory.

In various aspects, processor 1086 includes or is connected to communication interface 1015 that is coupled via network link 1016 (shown in phantom) to network 1050. For example, communication interface 1015 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WiFi or GSM. Communication interface 1015 sends and receives electrical, electromagnetic or optical signals that carry digital or analog data streams representing various types of information across network link 1016 to network 1050. Network link 1016 can be connected to network 1050 via a switch, gateway, hub, router, or other networking device.

Processor 1086 can send messages and receive data, including program code and imaging data, through network 1050, network link 1016 and communication interface 1015. For example, a server can store requested code for an application program (e.g., a JAVA applet) on a tangible non-volatile computer-readable storage medium to which it is connected. The server can retrieve the code from the medium and transmit it through network 1050 to communication interface 1015. The received code can be executed by processor 1086 as it is received, or stored in data storage system 1040 for later execution.

Data storage system 1040 can include or be communicatively connected with one or more processor-accessible memories configured to store information. The memories can be, e.g., within a chassis or as parts of a distributed system. The phrase “processor-accessible memory” is intended to include any data storage device to or from which processor 1086 can transfer data (using appropriate components of peripheral system 1020), whether volatile or nonvolatile; removable or fixed; electronic, magnetic, optical, chemical, mechanical, or otherwise. Exemplary processor-accessible memories include but are not limited to: registers, floppy disks, hard disks, tapes, bar codes, Compact Discs, DVDs, read-only memories (ROM), erasable programmable read-only memories (EPROM, EEPROM, or Flash), and random-access memories (RAMs). One of the processor-accessible memories in the data storage system 1040 can be a tangible non-transitory computer-readable storage medium, i.e., a non-transitory device or article of manufacture that participates in storing instructions that can be provided to processor 1086 for execution.

In an example, data storage system 1040 includes code memory 1041, e.g., a RAM, and disk 1043, e.g., a tangible computer-readable rotational storage device such as a hard drive. Computer program instructions are read into code memory 1041 from disk 1043. Processor 1086 then executes one or more sequences of the computer program instructions loaded into code memory 1041, as a result performing process steps described herein. In this way, processor 1086 carries out a computer implemented process. For example, steps of methods described herein, blocks of the flowchart illustrations or block diagrams herein, and combinations of those, can be implemented by computer program instructions. Code memory 1041 can also store data, or can store only code.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects These aspects can all generally be referred to herein as a “service,” “circuit,” “circuitry,” “module,” or “system.”

Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into processor 1086 (and possibly also other processors), to cause functions, acts, or operational steps of various aspects herein to be performed by the processor 1086 (or other processor). Computer program code for carrying out operations for various aspects described herein may be written in any combination of one or more programming language(s), and can be loaded from disk 1043 into code memory 1041 for execution. The program code may execute, e.g., entirely on processor 1086, partly on processor 1086 and partly on a remote computer connected to network 1050, or entirely on the remote computer.

While examples, one or more representative embodiments and specific forms of the disclosure have been illustrated and described in detail in the drawings and foregoing description, the same is to be considered as illustrative and not restrictive or limiting. The description of particular features in one embodiment does not imply that those particular features are necessarily limited to that one embodiment. Some or all of the features of one embodiment can be used in combination with some or all of the features of other embodiments as would be understood by one of ordinary skill in the art, whether or not explicitly described as such. One or more exemplary embodiments have been shown and described, and all changes and modifications that come within the spirit of the disclosure are desired to be protected.

I/We claim:

1. A method of processing data generated by an imaging system, wherein the imaging system is operable to generate a velocity data set and a magnitude data set representative of a fluid flow, the method comprising:
(a) receiving the velocity data set from the imaging system;
(b) calculating a phase variation data set from a wrapped phase field data set associated with the velocity data set;
(c) calculating a phase difference uncertainty data set from the magnitude data set;
(d) using the phase variation data set and the phase difference uncertainty data set, performing a computational reconstruction of the phase field data set to generate an unwrapped phase data set;
(e) converting the unwrapped phase to a first velocity field data set; and
(f) outputting a resultant velocity field set based upon the first velocity field data set.

2. The method of claim 1, wherein outputting the resultant velocity field set includes transmitting the resultant velocity field set to a graphical display.

3. The method of claim 1, wherein performing the computational reconstruction of the phase field data set to generate the unwrapped phase data set includes performing a weighted least squares operation to generate the unwrapped phase data set.

4. The method of claim 1, wherein the phase variation data set includes a spatial phase variation component and a temporal phase variation component, wherein the spatial phase variation component is representative of the difference between two or more neighboring voxels and the temporal phase variation component is representative of the difference between two or more consecutive cardiac frames.

5. The method of claim 1, wherein converting the unwrapped phase to the first velocity field data set includes multiplying the unwrapped phase by (venc/π).

6. The method of claim 1, wherein calculating the phase variation data set from the wrapped phase field value includes applying the formula [symbol] = [symbol] (Δψ), wherein [symbol] is the phase variation data set, wherein Δψ is a spatial variation data set or a temporal variation data set of the wrapped phase field data set.

7. The method of claim 1, wherein calculating the phase difference uncertainty data set from the magnitude data set includes incorporating with the magnitude data set a divergence-free constraint of incompressible flow.

8. The method of claim 1, wherein outputting the resultant velocity field set based upon the first velocity field data set includes:
(a) calculating a second phase variation data set from a second wrapped phase field data set associated with the first velocity field data set;
(b) calculating a second phase difference uncertainty data set from a second magnitude data set associated with the first velocity field data set;
(c) using the second phase variation data set and the second phase difference uncertainty data set, performing a second computational reconstruction of the second phase field data set to generate a second unwrapped phase data set;
(d) converting the second unwrapped phase to a second velocity field data set; and
(e) outputting the resultant velocity field set based upon the second velocity field data set.

9. A method of processing data generated by an imaging system, wherein the imaging system is operable to generate a velocity data set and a magnitude data set representative of a fluid flow, the method comprising:
(a) receiving the velocity data set from the imaging system;
(b) performing an unwrapping routine, including:
(i) calculating a phase variation data set from a wrapped phase field data set associated with the velocity data set;
(ii) calculating a phase difference uncertainty data set from the magnitude data set;
(ii) using the phase variation data set and the phase difference uncertainty data set, performing a computational reconstruction of the phase field data set to generate an unwrapped phase data set;
(ii) converting the unwrapped phase to a velocity field data set; and
(c) replacing the velocity data set with the velocity field data set;
(d) repeating steps (b)-(c) between five to 10 times; and
(e) outputting a resultant velocity field set based upon the velocity field data set.

10. The method of claim 9, wherein outputting the resultant velocity field set includes transmitting the resultant velocity field set to a graphical display.

11. The method of claim 9, wherein performing the computational reconstruction of the phase field data set to generate the unwrapped phase data set includes performing a weighted least squares operation to generate the unwrapped phase data set.

12. The method of claim 9, wherein the phase variation data set includes a spatial phase variation component and a temporal phase variation component, wherein the spatial phase variation component is representative of the difference between two or more neighboring voxels and the temporal phase variation component is representative of the difference between two or more consecutive cardiac frames.

13. The method of claim 9, wherein converting the unwrapped phase to the velocity field data set includes multiplying the unwrapped phase by (venc/π).

14. The method of claim 9, wherein calculating the phase variation data set from the wrapped phase field value includes applying the formula [symbol] = [symbol] (Δψ), wherein [symbol] is the phase variation data set, wherein Δψ is a spatial variation data set or a temporal variation data set of the wrapped phase field data set.

15. The method of claim 9, wherein calculating the phase difference uncertainty data set from the magnitude data set includes incorporating with the magnitude data set a divergence-free constraint of incompressible flow.

16. A post-processing system configured for use with a magnetic resonance imaging (MRI) based medical imaging system, wherein the MRI based medical imaging system is operable to generate a velocity data set and a magnitude data set representative of a fluid flow, the system comprising:
(a) at least one non-transitory processor-readable storage medium that stores at least one of processor-executable instructions or data; and
(b) at least one processor communicably coupled to the at least one non-transitory processor-readable storage medium, the at least one processor configured to:
(i) receive the velocity data set from the imaging system;
(ii) calculate a phase variation data set from a wrapped phase field data set associated with the velocity data set;
(iii) calculate a phase difference uncertainty data set from the magnitude data set;
(iv) use the phase variation data set and the phase difference uncertainty data set, performing a computational reconstruction of the phase field data set to generate an unwrapped phase data set;
(v) convert the unwrapped phase to a velocity field data set; and
(vi) output a resultant velocity field set based upon the velocity field data set; and
(c) a display device configured to receive and display the resultant velocity field set.

17. The system of claim 16, wherein performing the computational reconstruction of the phase field data set to generate the unwrapped phase data set includes performing a weighted least squares operation to generate the unwrapped phase data set.

18. The system of claim 16, wherein the phase variation data set includes a spatial phase variation component and a temporal phase variation component, wherein the spatial phase variation component is representative of the difference between two or more neighboring voxels and the temporal phase variation component is representative of the difference between two or more consecutive cardiac frames.

19. The system of claim 16, wherein calculating the phase variation data set from the wrapped phase field value includes applying the formula [symbol] = [symbol] (Δψ), wherein [symbol] is the phase variation data set, wherein Δψ is a spatial variation data set or a temporal variation data set of the wrapped phase field data set.

20. The system of claim 16, wherein calculating the phase difference uncertainty data set from the magnitude data set includes incorporating with the magnitude data set a divergence-free constraint of incompressible flow.
